(19) United States
(12) Patent Application Publication
(10) Pub. No.: US 2007/0181425 A1
(43) Pub. Date: Aug. 9, 2007

(54) Glucometer pack for communication device

(75) Inventor: Steven Kim, Palisades Park, NJ (US)

Correspondence Address: SEYFARTH SHAW, LLP, 815 CONNECTICUT AVENUE, N.W., SUITE 500, WASHINGTON, DC, 20006, US

(73) Assignee: Healthpia America, Palisades Park, NJ (US)

(21) Appl. No.: 11/348,342

(22) Filed: Feb. 7, 2006

(51) Int. Cl. C12Q 1/00; C12Q 1/68

(52) U.S. Cl. 204/403.11; 422/50

(57) ABSTRACT

A glucometer pack for a communication device includes a glucometer having a test strip receiver for measuring blood sugar and a back cover for the communication device, the back cover containing the glucometer and having an opening through which the test strip receiver of the glucometer can receive testing strips, wherein the back cover is configured to retain a battery of the communication device and attach to the communication device so as to position the battery to contact the power terminals of the communication device.

### BACKGROUND OF THE INVENTION

1. Field of the Invention

The present invention relates to a glucometer pack, and more particularly, to a glucometer pack for a communication device. Although the present invention is suitable for a wide scope of applications, it is particularly suitable as an add-on glucometer for a mobile phone.

2. Discussion of the Related Art

In general, a glucometer is a device for measuring a blood sugar level. Typically, a diabetic uses a glucometer to measure their blood sugar level. Repeated testing throughout the day is the best way for a diabetic to monitor their blood sugar level. Accordingly, there is a need for a glucometer that is portable and that can conveniently track the blood sugar level of a user.

FIG. 1a is a side view of a related art mobile phone having a regular battery pack. As shown in FIG. 1b, the mobile phone 1 includes a body 2, an antenna 4 protruding from the body 2, a data port 5 on the side of the body 2 and a flip-cover 6 that contains the display screen (not shown) of the mobile phone 1. The regular battery pack 10 is attached to the mobile phone 1. The regular battery pack 10 includes a regular battery 13 and a back cover 12 attached to the body 2 of the mobile phone 1 for holding a regular battery 13 in position.

Nearly all mobile phone devices include a rechargeable battery. The rechargeable battery powers the phone and keeps the phone operational for an extended period time. Some rechargeable batteries can keep a phone on in a stand-by mode for several days or keep a mobile phone communicating for several hours before the battery is completely drained. To further increase communication time and/or stand-by time, some models of mobile phones have an extended battery pack that can be used instead of the regular battery pack.

FIG. 1b is a side view of a related art mobile phone having an extended battery pack. As shown in FIG. 1b, the extended battery pack 11 has an extended battery 17 that is larger than the regular battery 13 shown in FIG. 1a. Further, an enlarged cover 18, which holds the extended battery 17 in position, is larger than the cover 12 in FIG. 1a. As can be seen by comparing FIG. 1a and FIG. 1b, the larger extended battery 18 increases the overall size of the mobile phone. However, the increased overall size does preclude the mobile phone from being used with the mobile phone cases or holders for the mobile phone. Many mobile phone cases or holders are designed to accommodate phones with either a regular battery pack or an extended battery pack. Some mobile phone holders are specifically designed for a particular model of mobile phone that has an extended battery pack.

FIG. 1c is a view of a back side of the related art mobile phone shown in FIG. 1a and a related art glucometer being attached to the side of the related art mobile phone. As shown in FIG. 1c, the mobile phone includes a body 2, a back cover 12 attached to the body 2 for covering a battery 13, an antenna 4 protruding from the body, and a data port 5 on the side of the body 2. The data port 5, such as universal serial bus (USB), allows the mobile phone to input/output data from an external device as well as to provide power to an external device.

Referring to FIG. 1c, an external glucometer 20 includes a slit 24 for receiving a measuring strip 26. When the measuring strip 26 is impregnated with a body fluid, for example, a drop of blood, the external glucometer 20 can measure the glucose level in the fluid. A data port connector 22 is provided at a side of the external glucometer 20. The data port connector 22 is insertable into the data port 5 of the mobile phone. The external glucometer 20 can be powered through the data port 5. Moreover, measurement data from the glucometer 20 can be downloaded to the mobile phone for communication to a health care provider.

The related art external glucometer hangs out from the side of a mobile phone. Thus, the related art external glucometer can easily become detached from the mobile phone. Further, the related art external glucometer can not be left attached to the mobile phone since the related art external glucometer will get in the way of everyday use and carry of the mobile phone. Accordingly, the related art external glucometer has to be stored separately from the mobile phone when not in use on the mobile phone. An internal glucometer can be built within the body of a mobile phone but then diabetics will have a limited choice as to which mobile phones they can use.

### SUMMARY OF THE INVENTION

Accordingly, the present invention is directed to a glucometer pack for a communication device that substantially obviates one or more of the problems due to limitations and disadvantages of the related art.

An object of the present invention is to provide a glucometer for a mobile phone while maintaining typical dimensions of the mobile phone.

Another object of the present invention is to provide of a glucometer which can be used with a variety of mobile phones.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, a glucometer pack for a communication device includes a glucometer having a test strip receiver for measuring blood sugar and a back cover for the communication device, the back cover containing the glucometer and having an opening through which the test strip receiver of the glucometer can receive testing strips, wherein the back cover is configured to retain a battery of the communication device and attach to the communication device so as to position the battery to contact the power terminals of the communication device.

In another aspect, a glucometer pack for a communication device includes a glucometer having a test strip receiver for measuring blood sugar, a battery for the communication device, and a back cover for the communication device, the back cover having an opening through which the test strip receiver of the glucometer can receive testing strips, wherein the back cover contains the glucometer and retains the battery.

In yet another aspect, a glucometer pack on a communication device includes a glucometer having a test strip receiver for measuring blood sugar, a battery for the communication device, and a back cover for the communication device, the back cover containing the glucometer and having an opening through which the test strip receiver of the glucometer can receive testing strips, wherein the back cover retains the battery and is attached to the communication device so as to position the battery to contact the power terminals of the communication device.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1a is a side view of a related art mobile phone having a regular battery pack.

FIG. 1b is a side view of a related art mobile phone having an extended battery pack.

FIG. 1c is a view of a back side of the related art mobile phone shown in FIG. 1a and a related art glucometer being attached to the side of the related art mobile phone.

FIG. 2a is a side view of a glucometer pack on a related art mobile phone in accordance with an embodiment of the present invention.

FIG. 2b is a back side view of both the glucometer pack and the related art mobile phone shown in FIG. 2a.

FIG. 2c is a top side view of both the glucometer pack and the related art mobile phone shown in FIG. 2a.

FIG. 3a is a top side view of a glucometer pack on a related art mobile phone in which the retractable connector of the glucometer pack is extended.

FIG. 3b is a back side view of both the glucometer pack and the related art mobile phone of FIG. 3a in which the retractable connector of the glucometer pack is extended.

FIG. 4a is a back side view of a glucometer pack on a related art mobile phone in which the retractable connector of the glucometer pack is connected to a data port of a mobile phone.

FIG. 4b is a top side view of the glucometer pack and the related art mobile phone shown of FIG. 4a in which the retractable connector of the glucometer pack is connected to a data port of a mobile phone.

FIG. 5 is a top side view of a glucometer pack on a related art mobile phone in which a connector of a glucometer pack is connected to a data port of a mobile phone in accordance with an embodiment of the present invention.

FIG. 6a is a side view of a glucometer pack on a related art mobile phone in which a data connector of the glucometer is connected to a battery/data port of a mobile phone in accordance with an embodiment of the present invention.

FIG. 6b is a top side view of both the glucometer pack and the related art mobile phone shown of FIG. 6a in which a data connector of the glucometer is connected to a battery/data port of the mobile phone.

FIG. 7 is a side view of a glucometer pack on a related art mobile phone in which the glucometer can wirelessly communicate with the mobile phone in accordance with an embodiment of the present invention.

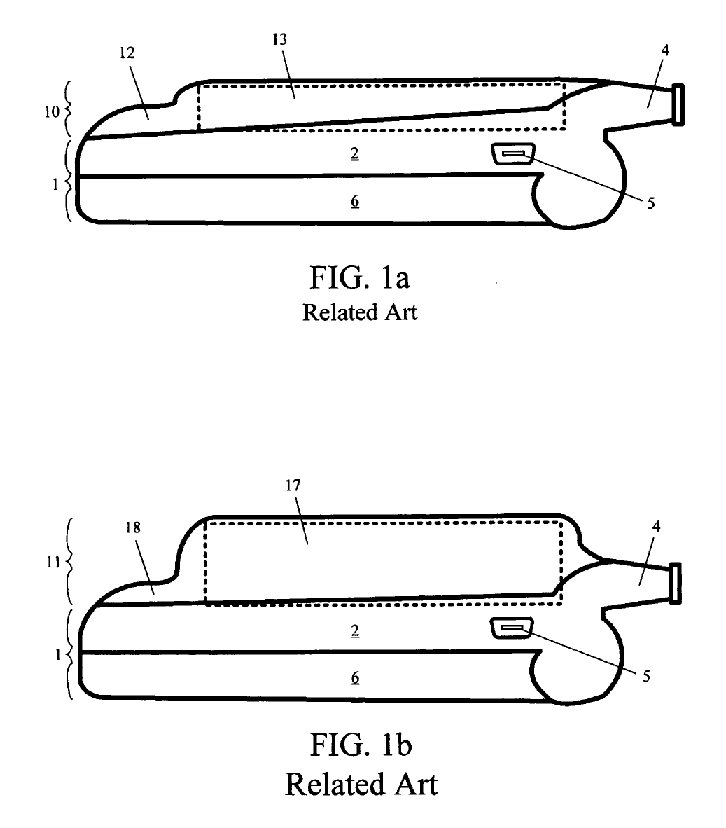

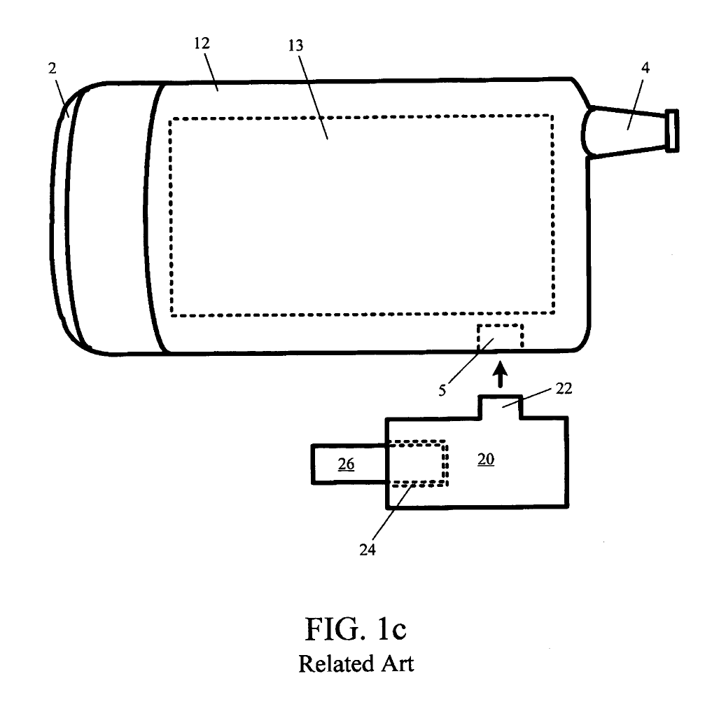

### DETAILED DESCRIPTION OF THE PREFERRED EMBODIMENTS

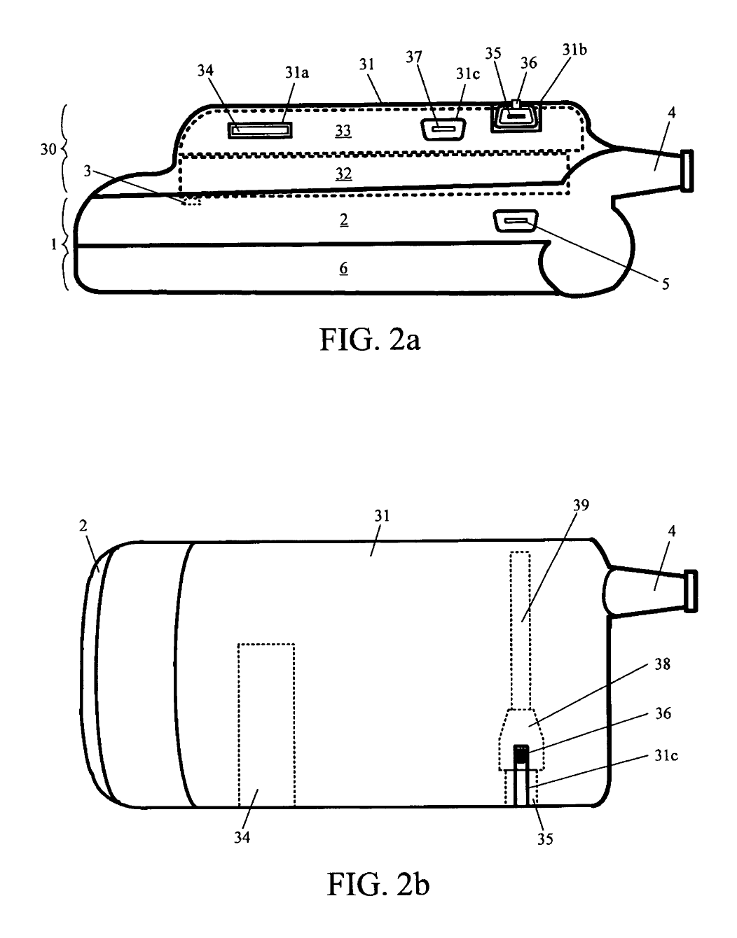

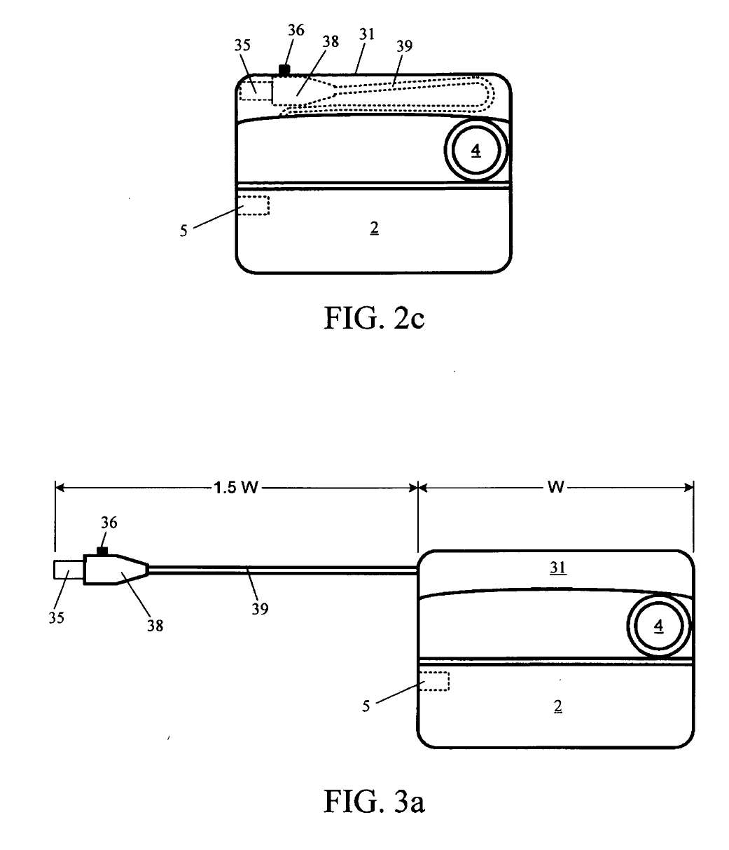

Reference will now be made in detail to the preferred embodiments of the present invention, examples of which are illustrated in the accompanying drawings. Like elements throughout the drawings are designated with the same reference numerals. Embodiments of the present invention will be explained with reference to FIGS. 2a, 2b, 2c, 3a, 3b, 4a, 4b, 5, 6a, 6b and 7.

FIG. 2a is a side view of a glucometer pack on a related art mobile phone in accordance with an embodiment of the present invention. As shown in FIG. 2a, the mobile phone 1 includes a body 2, an antenna 4 protruding from the body 2, a mobile phone data port 5 on the side of the body 2 and a flip-cover 6 containing a display screen (not shown) of the mobile phone 1. A glucometer pack 30 is attached to the mobile phone 1. The glucometer pack 30 includes a back cover 31, a standard size battery 32 and an external glucometer 33. More specifically, the extended back cover contains the external glucometer 33 and retains the battery 32. The extended back cover 31 can be attached to the body 2 of the mobile phone 1 so that the battery 32 is held in the proper position to contact the power terminals 3 of the mobile phone 1. Thus, the extended back cover 31 is configured to retain a battery 32 of the mobile phone 1 and attached to the mobile phone 1 so as to position the battery 32 to contact the power terminals 3 of the mobile phone 1.

Different types of mobile phones can have different types of extended back covers as well as different types of batteries. The external glucometer 33 can be used within a plurality of different types of extended back covers and with different types of batteries. The extended back covers in embodiments of the present invention are designed for different types of phones such that an attached extended back cover for a particular phone contains the external glucometer and retains the battery for that particular type of mobile phone.

As shown in FIG. 2a, the extended back cover 31 includes a test strip opening 31a through which a test strip receiver 34 of the external glucometer 33 can receive a testing strip (not shown in FIG. 2a). Further, the extended back cover 31 can include a data connector opening 31b for a retractable data connector 35 that is wired to the external glucometer 33. The retractable data connector 35 includes a lead 39 extending from the external glucometer, a plug body for insertion into a data port and a tab 36 on the plug body. The external glucometer can be connected to a mobile phone data port 5 on the side of the body 2 of the mobile phone 1 via the retractable data connector 35. By connecting the retractable data connector 35 to the mobile phone data port 5 on the side of the body 2 of the mobile phone 1, power can be provided to the external glucometer 33 and data can also be transferred between the mobile phone 1 and the external glucometer 33. The connection of the retractable data connector 35 to the mobile phone 35 can also enable the operation of a glucometer pack data port 37 within a data port opening 31c in the extended back cover 31 such that an external device can be connected to the mobile phone 1 through the glucometer pack data port 37.

FIG. 2b is a back side view of both the glucometer pack and the related art mobile phone shown in FIG. 2a. As shown in FIG. 2b, the retractable data connector 35 can be stored such that it is recessed within the back cover 31. The tab 36 on the plug body 38 fits into a slot 31d in the back cover when the retractable data connector 35 is recessed within the extended back cover 31. The retractable data connector 35 can be removed from its stored position by pushing the tab 36 such that the retractable data connector 35 is slid out of the data connector opening 31b.

FIG. 2c is a top side view of both the glucometer pack and the related art mobile phone shown in FIG. 2a. As shown in FIG. 2C, the lead 39 of the retractable data connector 35 can be coiled within the extended back cover 31. Thus, the lead 39 can be kept out of the way when the glucometer is not in use but yet has sufficient length to easily connect to the mobile phone data port 5 when the external glucometer 33 is to be used.

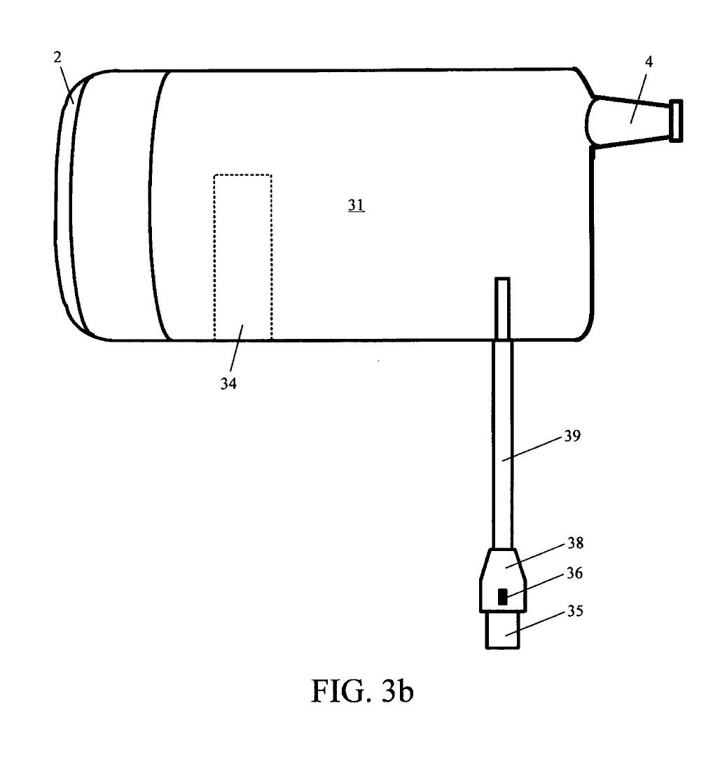

FIG. 3a is a top side view of a glucometer pack on a related art mobile phone in which the retractable connector of the glucometer pack is extended. FIG. 3b is a back side view of both the glucometer pack and the related art mobile phone of FIG. 3a in which the retractable connector of the glucometer pack is extended. As shown in FIGS. 3a and 3b, the lead 39 of the retractable data connector 35 has sufficient length for use with the mobile phone data port 5 on the body 2 of the mobile phone 1.

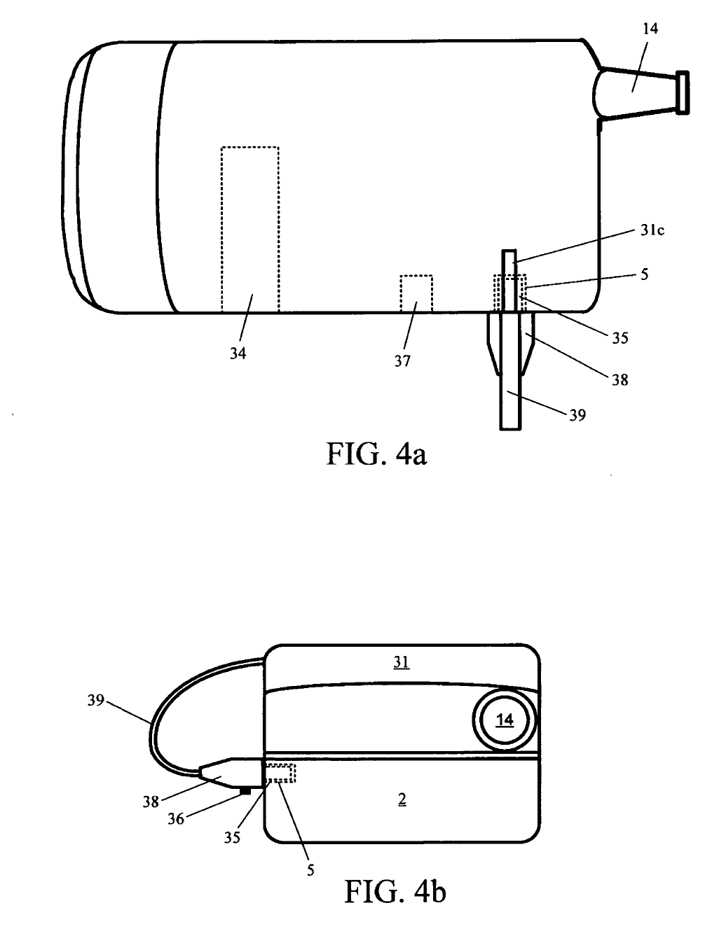

FIG. 4a is a back side view of a glucometer pack on a related art mobile phone in which the retractable connector of the glucometer pack is connected to a data port of a mobile phone. FIG. 4b is a top side view of the glucometer pack and the related art mobile phone shown of FIG. 4a in which the retractable connector of the glucometer pack is connected to a data port of a mobile phone. As shown in FIG. 4b, the retractable data cable 35 can extend out about 1.5 times the width W of the mobile phone 1. The extension length of the retractable data connector 35 increase the flexibility of the glucometer pack in that the glucometer pack can be used with mobile phones in which the mobile phone data port of the mobile phone is not immediately adjacent to the data connector opening in the back cover of glucometer pack.

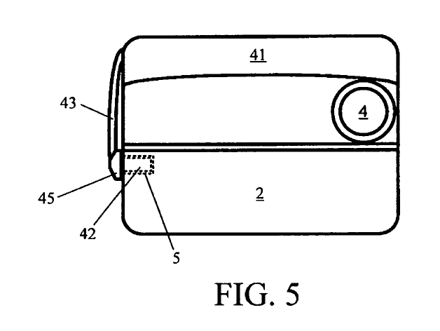

FIG. 5 is a top side view of a glucometer pack on a related art mobile phone in which a connector of a glucometer pack is connected to a data port of a mobile phone in accordance with an embodiment of the present invention. As shown in FIG. 5, a data connector 42 of an external glucometer can just have a lead 43 that extends from an extended back cover 41 to a low-profile plug body 43 instead of a connector that is a retractable data connector. The lead 43 should have sufficient length for the data connector 42 to be connected to the mobile phone data port 5 but yet keeps the lead 43 close to the body 2 of the mobile phone 1 so that the lead 43 is not obtrusive.

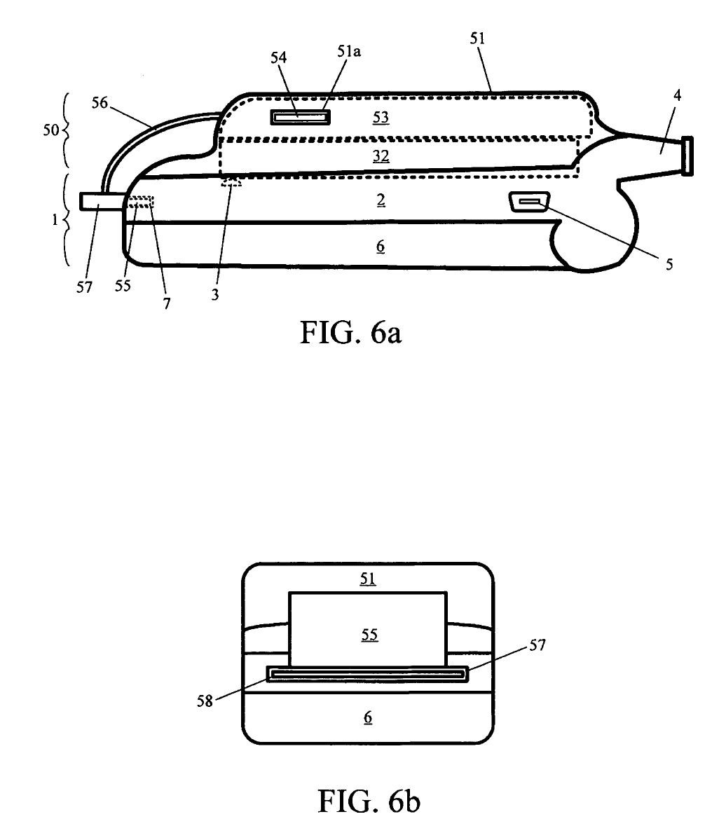

FIG. 6a is a side view of a glucometer pack on a related art mobile phone in which a data connector of the glucometer is connected to a battery/data port of a mobile phone in accordance with an embodiment of the present invention. FIG. 6b is a top side view of both the glucometer pack and the related art mobile phone shown of FIG. 6a in which a data connector of the glucometer is connected to a battery/data port of mobile phone. As shown in FIG. 6a, a glucometer pack 50 is attached to the mobile phone 1. The glucometer pack 50 includes a extended back cover 51, a standard size battery 32 and an external glucometer 53. More specifically, the extended back cover 51 contains the external glucometer 53 and retains the battery 32. The back cover 51 can be attached to the body 2 of the mobile phone 1 so that the battery 32 is held in the proper position to contact the power terminals 3 of the mobile phone 1. Thus, the extended back cover 51 is configured to retain a battery 32 of the mobile phone 1 and attached to the mobile phone 1 so as to position the battery 32 to contact the power terminals 3 of the mobile phone 1.

Referring to FIG. 6a, the glucometer pack 50 includes an extended back cover 51 with a test strip opening 51a through which a test strip receiver 54 of the external glucometer 53 can receive a test strip (not shown in FIG. 6a). Further, the extended back cover 51 can include a data connector 55 that is wired to the external glucometer 53. The data connector 55 can be connected to a mobile phone data/battery port 7 on the bottom side of the body 2 of the mobile phone 1. The data connector 55 can be a wire ribbon 56 that extends from the extended back cover 51 to a pass-through plug 57. The wiring ribbon 56 should have sufficient length for the data connector 55 to be connected to the mobile phone data/battery port 75 but yet keeps the wiring ribbon 56 close to the body 2 of the mobile phone so that the wiring ribbon 56 is not obtrusive.

The pass-through plug 57 has one end that plugs into the mobile phone data/battery port 7 and another end with a receptacle 58 that is a similar configuration to the mobile phone data/battery port 7. Thus, the pass-through plug 57 can be plugged into the mobile phone data/battery port 7 so that the glucometer pack 50 can communicate with the mobile phone 1 while another device is plugged into the receptacle 58 of the pass-through plug 57. For example, the receptacle of the pass-through plug can be connected to a battery charger while the pass-through plug is connected to the mobile phone data/battery port such that the battery can be charged while the glucometer pack is connected. In another example, the receptacle of the pass-through plug can be connected to a laptop computer while the pass-through plug is connected to the mobile phone data/battery port such that the laptop computer can transmit data wirelessly via the mobile phone while the glucometer pack is connected.

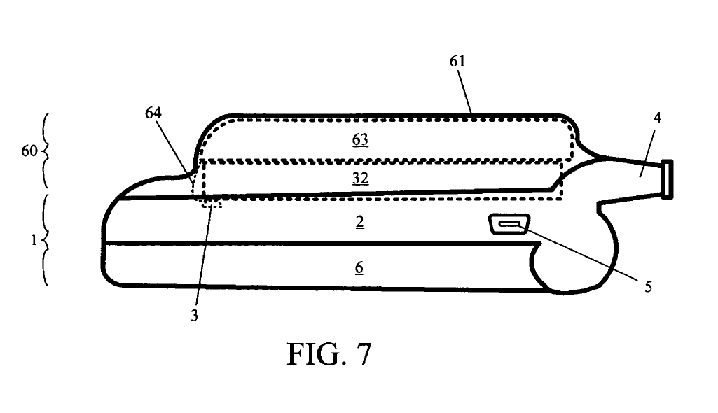

FIG. 7 is a side view of a glucometer pack on a related art mobile phone in which the glucometer can wirelessly communicate with the mobile phone in accordance with an embodiment of the present invention. The previous described embodiments of FIGS. 2a, 2b, 2c, 3a, 3b, 4a, 4b, 5, 6a and 6b show a wired connection via data connector between the mobile phone and the external glucometer within the glucometer pack. In the alternative, the data connector can be omitted and the external glucometer can communicate data with the mobile phone via wireless technology, such as bluetooth. When the external glucometer is configured to communicate wirelessly with the mobile phone, power is provided to the external glucometer via direct contact to the battery within the glucometer pack.

As shown in FIG. 7, a glucometer pack 60 is attached to the mobile phone 1. The glucometer pack 60 includes an extended back cover 61, a standard size battery 32 and an external glucometer 63 having wireless communication capability. More specifically, the extended back cover 61 contains the external glucometer 63 and retains battery 32. The back cover 61 can be attached to the body 2 of the mobile phone 1, so that the battery 32 is held in the proper position to contact the power terminals 3 of the mobile phone 1. Thus, the extended back cover 61 is configured to retain a battery 32 of the mobile phone 1 and attach to the mobile phone 1 so as to position the battery 32 to contact the power terminals 3 of the mobile phone 1. Further, the external glucometer 63 is connected to the battery through battery contact wires 64.

The glucometer pack in embodiments of the present invention can be used with other electronic devices that have a communication function. For instance, the glucometer pack in embodiments of the present invention can be used with a personal digital assistant (PDA), music player or a gaming device. Most digital devices that have a display and a battery compartment can have their battery cover reconfigured so as to include a glucometer in which testing results of the glucometer are displayed on the display screen of the digital device via a data port on the digital device.

The external glucometer in the glucometer pack can also be used separately from a mobile phone. For example, the non-retractable and retractable data connectors in embodiments of the present invention can be a mini universal serial bus (USB), which can connect to a personal computer. In the alternative, the non-retractable and retractable data connectors in embodiments of the present invention can be regular USB, firewire or other types of communication protocols. Thus, the glucometer in the glucometer pack can be used with a personal computer instead of a mobile phone. When the external glucometer in the glucometer pack uses wireless technology, such as bluetooth, to communicate, the external glucometer in the glucometer pack can communicate wirelessly with a personal computer having the wireless technology.

It will be apparent to those skilled in the art that various modifications and variations can be made in the glucometer pack for a communication device of the present invention without departing from the spirit or scope of the invention. Thus, it is intended that the present invention cover the modifications and variations of this invention provided they come within the scope of the appended claims and their equivalents.

What is claimed is:

1. A glucometer pack for a communication device, comprising:
a glucometer having a test strip receiver for measuring blood sugar; and
a back cover for the communication device, the back cover containing the glucometer and having an opening through which the test strip receiver of the glucometer can receive testing strips,
wherein the back cover is configured to retain a battery of the communication device and attach to the communication device so as to position the battery to contact the power terminals of the communication device.

2. The glucometer pack of claim 1, further comprising a data connector wired to the glucometer.

3. The glucometer pack of claim 2, wherein the data connector is retractable into the back cover.

4. The glucometer pack of claim 3, wherein the data connector includes a lead extending from the glucometer, a plug body for insertion into a data port and a tab on the plug body for removing the data connector from within the back cover.

5. The glucometer pack of claim 2, wherein the data connector includes a pass-through plug.

6. The glucometer pack of claim 1, wherein the glucometer is configured to wirelessly transmit data.

7. The glucometer pack of claim 1, further comprising a data port within a data port opening in the back cover.

8. A glucometer pack for a communication device, comprising:
a glucometer having a test strip receiver for measuring blood sugar;
a battery for the communication device; and
a back cover for the communication device, the back cover having an opening through which the test strip receiver of the glucometer can receive testing strips,
wherein the back cover contains the glucometer and retains the battery.

9. The glucometer pack of claim 8, further comprising a data connector that is retractable into the back cover.

10. The glucometer pack of claim 8, further comprising a data connector that includes a pass-through plug.

11. The glucometer pack of claim 8, wherein the glucometer is configured to wirelessly transmit data.

12. The glucometer pack of claim 11, wherein the glucometer is directly connected to the battery within the back cover.

13. The glucometer pack of claim 8, further comprising a data port within a data port opening in the back cover.

14. A glucometer pack on a communication device, comprising:
a glucometer having a test strip receiver for measuring blood sugar;
a battery for the communication device; and
a back cover for the communication device, the back cover containing the glucometer and having an opening through which the test strip receiver of the glucometer can receive testing strips,
wherein the back cover retains the battery and is attached to the communication device so as to position the battery to contact the power terminals of the communication device.

15. The glucometer pack of claim 14, further comprising a data connector wired to the glucometer connected to a data port of the communication device.

16. The glucometer pack of claim 15, wherein the data connector is retractable into the back cover.

17. The glucometer pack of claim 15, wherein the data connector includes a pass-through plug.

18. The glucometer pack of claim 14, wherein the glucometer is configured to wirelessly transmit data to the communication device.

19. The glucometer pack of claim 18, wherein the glucometer is directly connected to the battery within the back cover.

20. The glucometer pack of claim 14, further comprising data port within a data port opening in the back cover.
